(19) United States
(12) Patent Application Publication
(10) Pub. No.: US 2024/0415877 A1
(43) Pub. Date: Dec. 19, 2024

(54) METHODS OF TREATING NON-BLEEDING WOUNDS, CHRONIC WOUNDS, INFLAMMATORY PAIN AND NOCICEPTIVE PAIN

(71) Applicant: Ariel Scientific Innovations Ltd., Ariel (IL)

(72) Inventor: Danny BARANES, Ramat HaSharon (IL)

(73) Assignee: Ariel Scientific Innovations Ltd., Ariel (IL)

(21) Appl. No.: 18/689,875

(22) PCT Filed: Sep. 8, 2022
(86) PCT No.: PCT/IL2022/050979

§ 371 Date: Mar. 7, 2024

Related U.S. Application Data
(60) Provisional application No. 63/241,583, filed on Sep. 8, 2021.

(51) Int. Cl. A61K 33/10; A61K 38/02; A61P 17/02

(52) U.S. Cl. CPC A61K 33/10; A61K 38/02; A61P 17/02

(57) ABSTRACT

A method of treating a chronic wound, a non-bleeding wound, an inflammatory pain or a nociceptive pain in a subject in need thereof. The method comprising administering to the subject an effective amount of a composite material comprising a calcium carbonate-containing material, and an associating moiety being associated with the calcium carbonate-containing material.

### RELATED APPLICATION

This application claims priority from U.S. Provisional Patent Application No. 63/241,583 filed Sep. 8, 2021 and is hereby incorporated by reference in its entirety.

### FIELD AND BACKGROUND OF THE INVENTION

The present invention, in some embodiments thereof, relates to methods of treating non-bleeding wounds, chronic wounds, inflammatory pain and nociceptive pain.

Wounds, especially chronic wounds, are a major source of suffer, pain, disability and even death, with a huge economical cost. Wound healing is therefore one of the central goals of modern medicine, involving basic, biotechnological, and clinical research. A large range of solutions to wound healing are in the market, mainly dressings, that cause recovery by eliciting wound drying, or vice a versa keeping it moist or activating a range of anti-inflammatory or tissue regenerating molecules using specific drugs for each action. However, to date there is no robust solution for wound healing.

### SUMMARY OF THE INVENTION

According to an aspect of some embodiments of the present invention there is provided a method of treating a chronic wound in a subject in need thereof, the method comprising administering to the subject an effective amount of a composite material comprising a calcium carbonate-containing material, and an associating moiety being associated with the calcium carbonate-containing material.

According to an aspect of some embodiments of the present invention there is provided a method of treating a non-bleeding wound, the method comprising administering to the subject an effective amount of a composite material comprising a calcium carbonate-containing material, and an associating moiety being associated with the calcium carbonate-containing material.

According to an aspect of some embodiments of the present invention there is provided a method of treating or preventing an inflammatory pain or a nociceptive pain the method comprising administering to the subject an effective amount of a composite material comprising a calcium carbonate-containing material, and an associating moiety being associated with the calcium carbonate-containing material.

According to some embodiments of the invention, the inflammatory or a nociceptive pain is associated with a bruise, a burn, a cut, a bone fracture, muscle overuse and/or a joint damage.

According to some embodiments of the invention, the wound is a skin wound.

According to some embodiments of the invention, the wound is a diabetic wound.

According to some embodiments of the invention, the subject is a human subject.

According to some embodiments of the invention, the subject is a non-human subject.

According to some embodiments of the invention, the administering comprises topical administration.

According to some embodiments of the invention, the administering comprises multiple administrations.

According to some embodiments of the invention, the multiple administrations are affected over a period of at least 7 days.

According to some embodiments of the invention, the composite is formulated in a form of a powder, a gel, a spray, a foam, a mousse, an ointment, a paste, a lotion, a gauze, a wound dressing, a suspension, an adhesive bandage, a non-adhesive bandage, a wipe, a gauze, a pad, and a sponge.

According to some embodiments of the invention, the composite further comprises a citrate in chemical association with the calcium carbonate, and/or with the association moiety.

According to some embodiments of the invention, the associating moiety is a positively-charged moiety at physiological pH.

According to some embodiments of the invention, the association moiety is a polymeric moiety.

According to some embodiments of the invention, the association moiety has a molecular weight in a range of 10 to 100 kDa.

According to some embodiments of the invention, the association moiety has a molecular weight of at least 300 kDa.

According to some embodiments of the invention, the association moiety is a biocompatible moiety.

According to some embodiments of the invention, the association moiety is a polypeptide.

According to some embodiments of the invention, the polypeptide comprises at least one amino acid residue that is positively charged at physiological pH.

According to some embodiments of the invention, the polypeptide essentially consists of amino acid residues that are positively charged at physiological pH.

According to some embodiments of the invention, the polypeptide is or comprises a polylysine.

According to some embodiments of the invention, the polylysine is selected poly-D-lysine, poly-L-lysine and poly-ε-lysine.

According to some embodiments of the invention, the polypeptide is or comprises collagen.

According to some embodiments of the invention, the association moiety is or comprises lysine.

According to some embodiments of the invention, the lysine is selected from L-lysine, D-lysine and ε-Lysine.

According to some embodiments of the invention, the calcium carbonate-containing material comprises crystalline calcium carbonate.

According to some embodiments of the invention, the calcium carbonate-containing material comprises a coral exoskeleton.

According to some embodiments of the invention, the calcium carbonate-containing material comprises amorphous calcium carbonate (ACC).

According to some embodiments of the invention, the calcium carbonate-containing material is a particulate material.

According to some embodiments of the invention, the particulate material comprises particles having an average particle diameter in the range of from 0.1 micron to 10 millimeter, or from 0.1 micron to 1 millimeter, or from 0.1 micron to 500 microns, or from 0.5 microns to 500 microns, or from 1 micron to 500 microns, or from 5.0 microns to 500 microns.

According to some embodiments of the invention, the particulate material comprises particles having an average particle diameter in the range of from 0.1 micron to 100 microns, or from 0.1 microns to 50 microns.

According to some embodiments of the invention, the particulate material comprises particles having an average diameter in the range of from 100 microns to 10 millimeter, or from 100 microns to 1 millimeter.

According to some embodiments of the invention, a weight ratio of the citrate and the calcium carbonate-containing material ranges from 10:1 to 1:10, or from 5:1 to 1:5.

According to some embodiments of the invention, a weight ratio of the association moiety and the calcium carbonate-containing material ranges from 5000:1 to 250:1.

According to some embodiments of the invention, the method further comprises using a swelling polymeric moiety.

According to some embodiments of the invention, the swelling polymeric moiety is selected from alginate, chitosan, collagen and a poly(alkylene glycol).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an image showing that CPC induces wound healing of chronic wounds in horses.

FIG. 2 is an image showing that CPC induces wound healing of a chronic wound in a dog.

FIG. 3 is an image showing that CPC induces wound healing of a chronic wound in a cat.

FIG. 4 shows that CPC treats non-bleeding skin wounds in mice versus control non-treated wounds. (Upper left) Punch-made skin wounds. (Upper right) Treatment with CPC. (Bottom) Quantification of wound diameters during days following the injury.

### DESCRIPTION OF SPECIFIC EMBODIMENTS OF THE INVENTION

The present invention, in some embodiments thereof, relates to methods of treating non-bleeding wounds, chronic wounds, inflammatory pain and nociceptive pain.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst conceiving embodiments of the invention, the present inventors realized that a composite material comprising calcium carbonate, an association moiety and optionally citrate can be used to effectively treat non-bleeding wounds (e.g., acute), chronic wounds, inflammatory pain and nociceptive pain.

The effectivity of this composite material was demonstrated on a multiple of animal models with various types of wounds.

Without being bound by theory it is envisaged that:

1. The calcium carbonate component has a few functions:
it dries the wound by absorbing liquid;
it elevates the pH;
it reduces wound size by inhibiting pro-inflammatory mediators and reducing edema.
   2. Citrate causes calcium carbonate to release calcium ions which activate tissue recovery factors
   3. The association moiety such as the lysine promotes tissue recovery by deriving collagen formation

The combinations of at least two of the above (calcium carbone and association moiety, also termed as “CP”) or all three (which are termed as “CPC”) in one drug—reduce wound severity (decreased inflammation), stabilize wound microenvironment (elevation of pH and absorption of liquids) and accelerate repair (calcium release, lysine action). Finally, the combined pH elevation while reducing wound secretions (by water absorption and reducing edema) gives rise to a debridement-like process. Results provided in the Examples section which follows show that CPC causes rapid granulation, size and severity reduction, and restoration structure and function of the injured tissue in chronic wounds.

Thus, according to an aspect of the invention there is provided a method of treating a chronic wound in a subject in need thereof, the method comprising administering to the subject an effective amount of a composite material comprising a calcium carbonate-containing material, and an associating moiety being associated with the calcium carbonate-containing material.

Alternatively or additionally, there is provided a method of treating a non-bleeding wound, the method comprising administering to the subject an effective amount of a composite material comprising a calcium carbonate-containing material, and an associating moiety being associated with the calcium carbonate-containing material.

Alternatively or additionally, there is provided a method of treating or preventing inflammatory pain or nociceptive pain the method comprising administering to the subject an effective amount of a composite material comprising a calcium carbonate-containing material, and an associating moiety being associated with the calcium carbonate-containing material.

Alternatively or additionally, there is provided a composite material comprising a calcium carbonate-containing material, and an associating moiety being associated with the calcium carbonate-containing material for use in treating a chronic wound.

Alternatively or additionally, there is provided a composite material comprising a calcium carbonate-containing material, and an associating moiety being associated with the calcium carbonate-containing material for use in treating a non-bleeding wound.

Alternatively or additionally, there is provided a composite material comprising a calcium carbonate-containing material, and an associating moiety being associated with the calcium carbonate-containing material for use in treating or preventing inflammatory pain or nociceptive pain.

As used herein, the term “subject” refers to a human or non-human subject (i.e., veterinary use). General livestock or domesticated animals are contemplated herewith in a specific embodiment, such as mammals or non-mammals, including but not limited to a bird, a cow, a horse, a goat, a sheep, a pig, a dog, a cat, a chicken and a turkey. According to a specific embodiment, the subject in a human being at any age who suffer from a pathology that requires induction of wound healing or alleviation of inflammatory pain or nociceptive pain or is at risk for such a medical condition (e.g., inflammatory pain or nociceptive pain).

The term “treating” refers to inhibiting, preventing or arresting the development of a pathology (e.g., a wound and/or inflammatory pain or nociceptive pain) and/or causing the reduction, remission, or regression of a pathology (e.g., a wound and/or inflammatory pain or nociceptive pain). Those of skill in the art will understand that various methodologies and assays can be used to assess the development of a pathology, and similarly, various methodologies and assays may be used to assess the reduction, remission or regression of a pathology.

As used herein, the term “preventing” refers to keeping condition (e.g., inflammatory pain or nociceptive pain) from occurring in a subject who may be at risk for the condition, but has not yet been diagnosed as having the condition.

As used herein “a chronic wound” refers to a wound that does not heal in an orderly set of stages and in a predictable amount of time; wounds that do not heal within three months are considered chronic. Chronic wounds seem to be detained in one or more of the phases of wound healing. For example, chronic wounds often remain in the inflammatory stage for too long. To overcome that stage and jump-start the healing process a number of factors need to be addressed such as bacterial burden, necrotic tissue, and moisture balance of the whole wound. In acute wounds, there is a precise balance between production and degradation of molecules such as collagen; in chronic wounds this balance is lost and degradation plays too large a role.

Acute and chronic wounds are at opposite ends of a spectrum of wound-healing types that progress toward being healed at different rates.

According to a specific embodiment, the chronic wound is of the skin or any layer thereof.

Medical conditions which may be associated with chronic wounds include but are not limited to poor circulation, neuropathy, and difficulty moving, systemic illnesses, age, and repeated trauma. The genetic skin disorders collectively known as epidermolysis bullosa display skin fragility and a tendency to develop chronic, non-healing wounds. Comorbid ailments that may contribute to the formation of chronic wounds include vasculitis (an inflammation of blood vessels), immune suppression, pyoderma gangrenosum, and diseases that cause ischemia. Immune suppression can be caused by illnesses or medical drugs used over a long period, for example steroids. Emotional stress can also negatively affect the healing of a wound, possibly by raising blood pressure and levels of cortisol, which lowers immunity.

What appears to be a chronic wound may also be a malignancy; for example, cancerous tissue can grow until blood cannot reach the cells and the tissue becomes an ulcer. Cancer, especially squamous cell carcinoma, may also form as the result of chronic wounds, probably due to repetitive tissue damage that stimulates rapid cell proliferation.

Another factor that may contribute to chronic wounds is old age. The skin of older people is more easily damaged, and older cells do not proliferate as fast and may not have an adequate response to stress in terms of gene upregulation of stress-related proteins. In older cells, stress response genes are overexpressed when the cell is not stressed, but when it is, the expression of these proteins is not upregulated by as much as in younger cells.

Comorbid factors that can lead to ischemia are especially likely to contribute to chronic wounds. Such factors include chronic fibrosis, edema, sickle cell disease, and peripheral artery disease such as by atherosclerosis.

Repeated physical trauma plays a role in chronic wound formation by continually initiating the inflammatory cascade. The trauma may occur by accident, for example when a leg is repeatedly bumped against a wheelchair rest, or it may be due to intentional acts. Heroin users who lose venous access may resort to ‘skin popping’, or injecting the drug subcutaneously, which is highly damaging to tissue and frequently leads to chronic ulcers.

Periwound skin damage caused by excessive amounts of exudate and other bodily fluids can perpetuate the non-healing status of chronic wounds. Maceration, excoriation, dry (fragile) skin, hyperkeratosis, callus and eczema are frequent problems that interfere with the integrity of periwound skin. They can create a gateway for infection as well as cause wound edge deterioration preventing wound closure.

Since more oxygen in the wound environment allows white blood cells to produce ROS to kill bacteria, patients with inadequate tissue oxygenation, for example those who suffered hypothermia during surgery, are at higher risk for infection. The host's immune response to the presence of bacteria prolongs inflammation, delays healing, and damages tissue. Infection can lead not only to chronic wounds but also to gangrene, loss of the infected limb, and death of the patient.

Like ischemia, bacterial colonization and infection damage tissue by causing a greater number of neutrophils to enter the wound site. In patients with chronic wounds, bacteria with resistances to antibiotics may have time to develop. In addition, patients that carry drug resistant bacterial strains such as methicillin-resistant Staphylococcus aureus (MRSA) have more chronic wounds.

Chronic wounds also differ in makeup from acute wounds in that their levels of proteolytic enzymes such as elastase.^{[5]} and matrix metalloproteinases (MMPs) are higher, while their concentrations of growth factors such as Platelet-derived growth factor and Keratinocyte Growth Factor are lower. Since growth factors (GFs) are imperative in timely wound healing, inadequate GF levels may be an important factor in chronic wound formation. In chronic wounds, the formation and release of growth factors may be prevented, the factors may be sequestered and unable to perform their metabolic roles, or degraded in excess by cellular or bacterial proteases.

Chronic wounds such as diabetic and venous ulcers are also caused by a failure of fibroblasts to produce adequate ECM proteins and by keratinocytes to epithelialize the wound. Fibroblast gene expression is different in chronic wounds than in acute wounds.

Though all wounds require a certain level of elastase and proteases for proper healing, too high a concentration is damaging. Leukocytes in the wound area release elastase, which increases inflammation, destroys tissue, proteoglycans, and collagen, and damages growth factors, fibronectin, and factors that inhibit proteases. The activity of elastase is increased by human serum albumin, which is the most abundant protein found in chronic wounds. As such, chronic wounds with inadequate albumin are especially unlikely to heal.

Excess matrix metalloproteinases, which are released by leukocytes, may also cause wounds to become chronic. MMPs break down ECM molecules, growth factors, and protease inhibitors, and thus increase degradation while reducing construction, throwing the delicate compromise between production and degradation out of balance.

According to an embodiment of the invention the chronic wound is a venous ulcer.

Venous ulcers, which usually occur in the legs, account for about 70% to 90% of chronic wounds and mostly affect the elderly. They are thought to be due to venous hypertension caused by improper function of valves that exist in the veins to prevent blood from flowing backward.

According to an embodiment of the invention the chronic wound is a diabetic ulcer.

A major cause of chronic wounds, diabetes, is increasing in prevalence. Diabetics have a 15% higher risk for amputation than the general population due to chronic ulcers. Patients may not initially notice small wounds to legs and feet, and may therefore fail to prevent infection or repeated injury. Further, diabetes causes immune compromise and damage to small blood vessels, preventing adequate oxygenation of tissue, which can cause chronic wounds. Pressure also plays a role in the formation of diabetic ulcers.

According to an embodiment of the invention the chronic wound is a pressure ulcer.

Another leading type of chronic wounds is pressure ulcers, which usually occur in people with conditions such as paralysis that inhibit movement of body parts that are commonly subjected to pressure such as the heels, shoulder blades, and sacrum. Pressure ulcers are caused by ischemia that occurs when pressure on the tissue is greater than the pressure in capillaries, and thus restricts blood flow into the area. Muscle tissue, which needs more oxygen and nutrients than skin does, shows the worst effects from prolonged pressure. As in other chronic ulcers, reperfusion injury damages tissue.

According to other embodiments the chronic wound is a result of a radiation poisoning or ischemia.

Ischemia results from the dysfunction and, combined with reperfusion injury, causes the tissue damage that leads to the wounds.

As used herein “a non-bleeding wound” refers to an acute wound or a chronic wound with no hemorrhage.

According to a specific embodiment, the wound is an abrasion, a laceration or a puncture.

As used herein “a nociceptive pain” refers to a pain which happens when nociceptors detect something that can cause harm to the body, like a chemical, hot or cold temperature, or physical force. Nociceptors sense physical damage to the skin, muscles, bones or connective tissue in the body.

The nociceptive pain can be associated with either of a bruise, a burn, a cut, a bone fracture, a muscle overuse and/or a joint damage, each of which is contemplated herewith as if it were a separate embodiment. Nociceptive pain caused by a systemic disease such as cancer is also contemplated herewith.

As used herein, “an inflammatory pain” is caused by the direct or side effect of inflammation. Direct inflammatory pain can be derived by the action of bradykinin released by mast cells on peripheral nerve endings. Indirect inflammatory pain can be caused by edema generated by capillary expansion occurring and the injury site by histamine.

### The Composite Material:

As mentioned, treatment according to embodiments of the invention is effected using a composite material, which comprises a calcium carbonate-containing material, an lysine (or poly-D- or poly-L-lysine) being associated with the calcium carbonate-containing material and optionally citrate associated with the lysine (or poly-D- or poly-L-lysine).

By “associated with” it is meant physical and/or chemical association between the indicated components.

Thus, for example, the citrate and/or lysine (or poly-D- or poly-L-lysine) can be attached to the calcium carbonate-containing material, by interacting with the carbonate groups and/or the calcium cations via, e.g., covalent bonds, electrostatic interactions, hydrogen bonding, van der Waals interactions, donor-acceptor interactions, and/or cation-π interactions. These interactions lead to the chemical association between the components.

As an example, and without being bound by any particular theory, the citrate ions may be in chemical association with the positively charged calcium of the calcium carbonate, and/or with positively charged groups of the lysine (or poly-D- or poly-L-lysine). The carbonate groups of the calcium carbonate can also be in chemical association with positively charged groups of the association moiety, while the calcium can be in chemical association with negatively charged group of the association moiety.

Alternatively, the components can be attached to one another by physical association such as surface adsorption, encapsulation, entrapment, entanglement and the likes.

### The Calcium-Carbonate Containing Material:

Herein throughout, the phrase “calcium carbonate-containing material” describes a material, a substance or a composition-of-matter, which is substantially consisting of calcium carbonate, that it, which includes at least 50%, or at least 60%, or at least 70%, or at least 80%, or at least 90%, or at least 95%, or about 100%, by weight, calcium carbonate.

The term “calcium carbonate” as used herein, refers to the chemical compound CaCO₃. In some embodiments, the calcium carbonate is solid calcium carbonate, which can be in crystalline or amorphous form. As used herein, crystalline forms of calcium carbonate include aragonite, calcite, ikaite, vaterite and monohydrocalcite, all of which are encompassed. Other solid forms of calcium carbonate include amorphous calcium carbonate (ACC).

Calcium carbonate-containing material usable in the context of the present embodiments can be obtained or derived from natural sources (e.g., from living species or geological matter), or be synthetic (chemically synthesized). Commercially available forms of calcium carbonate are also encompassed.

Natural sources of calcium carbonate include, but are not limited to, rock formations, such as limestone, chalk, marble, travertine and tufa, as well as other geological matters. Calcium carbonate is also a principle structural component of many life forms, and thus can be obtained from, inter alia, corals, plankton, coralline algae, sponges, brachiopods, echinoderms, bryozoa, mollusks and other calcium carbonate-containing organisms.

In some of any of the embodiments described herein, the calcium carbonate-containing material comprises one or more forms of crystalline calcium carbonate.

In some of any of the embodiments described herein, the calcium carbonate-containing material comprises, or consists of, one or more forms of aragonite, calcite, ikaite, vaterite, and monohydrocalcite.

In some of any of the embodiments described herein, the calcium carbonate-containing material comprises aragonite. As used herein, the term “aragonite” refers to the crystalline form of calcium carbonate, which can be commonly found in as mineral deposits in caves and in oceans, and in the shells of mollusks and exoskeleton of cold and warm-water corals. The aragonite can be a geological aragonite or a biogenic aragonite (derived from living species such as corals or mollusks). Geological aragonite typically has a crystalline structure that is different from biogenic aragonite.

In some of any of the embodiments described herein, the calcium carbonate-containing material comprises calcite. As used herein, the term “calcite” refers to a crystalline form of calcium carbonate, differing from aragonite in its crystal lattice form, which can be obtained from sedimentary rocks and from the shells of some marine organisms.

In some of any of the embodiments described herein, the calcium carbonate-containing material comprises both aragonite and calcite.

In some of any of the embodiments described herein, the calcium carbonate-containing material (e.g., aragonite) comprises a coral exoskeleton. The term “coral exoskeleton”, as used herein, refers to the exoskeleton of marine madreporic corals or material derived therefrom. Natural coral (e.g., Porites) consists of a mineral phase, principally calcium carbonate, typically in the structural form of aragonite or calcite, with impurities, such as Sr, Mg and F ions, and an organic matrix. Thus, as used herein, “coral exoskeleton” includes calcium carbonate, e.g., in the form of aragonite or calcite, with or without additional components (minerals, organic and inorganic components) derived from or secreted by the living coral or life forms associated therewith.

The term “coral exoskeleton” is also referred to herein simply as “coral skeleton” or abbreviated as “CS”.

In some of any of the embodiments described herein, the calcium carbonate-containing material is derived from a coral and comprises a coral exoskeleton.

Coral exoskeleton can be a commercially available material (e.g., Biocoral™) and has been reported to be biocompatible and resorbable. Coral-derived material described as coralline HA prepared by hydrothermally converting the original calcium carbonate of the coral Porites in the presence of ammonium phosphate, maintaining the original interconnected macroporosity of the coral, is also commercially-available (Pro Osteon®, Interpore Cross). The high content calcium carbonate coral exoskeleton has been shown to be biocompatible and biodegradable at variable rates depending on porosity, the implantation site and the species.

In some of any of the embodiments described herein, the coral exoskeleton or materials comprising the same are derived from a coral. In some embodiments, the coral can comprise any species, including, but not limited to, Porites, Stylophora, Acropora, Millepora, or a combination thereof.

In some embodiments, the coral is from the Porites species. In some embodiments, the coral is Porites lutea.

In some embodiments, the coral is from the Acropora species. In some embodiments, the coral is Acropora grandis (which in one embodiment is very common, fast growing, and easy to culture). Acropora samples can be easily collected in sheltered areas of the coral reefs and/or can conveniently be cultured.

In some embodiments, the coral is from the Millepora species. In one embodiment, the coral is Millepora dichotoma. In one embodiment, the coral has a pore size of 150 microns and can be cloned and cultured, making Millepora useful in the compositions and methods of this invention.

In some embodiments, the coral is from the Stylophora species. Stylophora is a genus of colonial stony corals in the family Pocilloporidae, commonly known as cat's paw corals or birdsnest corals. In some embodiments, the coral is Stylophora subseriata.

In another embodiment, the coral can be from any one or more of the following species: Favites halicora; Goniastrea retiformis; Acanthastrea echinata; Acanthastrea hemprichi; Acanthastrea ishigakiensis; Acropora aspera; Acropora austera; Acropora sp. “brown digitate”; Acropora carduus; Acropora cerealis; Acropora chesterfieldensis; Acropora clathrata; Acropora cophodactyla; Acropora sp. “danai-like”; Acropora divaricata; Acropora donei; Acropora echinata; Acropora efflorescens; Acropora gemmifera; Acropora globiceps; Acropora granulosa; Acropora cf hemprichi; Acropora kosurini; Acropora cf loisettae; Acropora longicyathus; Acropora loripes; Acropora cf lutkeni; Acropora paniculata; Acropora proximalis; Acropora rudis; Acropora selago; Acropora solitaryensis; Acropora cf spicifera as per Veron; Acropora cf spicifera as per Wallace; Acropora tenuis; Acropora valenciennesi; Acropora vaughani; Acropora vermiculata; Astreopora gracilis; Astreopora myriophthalma; Astreopora randalli; Astreopora suggesta; Australomussa rowleyensis; Coscinaraea collumna; Coscinaraea crassa; Cynarina lacrymalis; Distichopora violacea; Echinophyllia echinata; Echinophyllia cf echinoporoides; Echinopora gemmacea; Echinopora hirsutissima; Euphyllia ancora; Euphyllia divisa; Euphyllia yaeyamensis; Favia rotundata; Favia truncatus; Favites acuticollis; Favities pentagona; Fungia granulosa; Fungia klunzingeri; Fungia mollucensis; Galaxea acrhelia; Goniastrea edwardsi; Goniastea minuta; Hydnophora pilosa; Leptoseris explanata; Leptoseris incrustans; Leptoseris mycetoseroides; Leptoseris scabra; Leptoseris yabei; Lithophyllon undulatum; Lobophyllia hemprichii; Merulina scabricula; Millepora dichotoma; Millepora exaesa; Millipora intricata; Millepora murrayensis; Millipore platyphylla; Monastrea curta; Monastrea colemani; Montipora caliculata; Montipora capitata; Montipora foveolata; Montipora meandrina; Montipora tuberculosa; Montipora cf vietnamensis; Oulophyllia laevis; Oxypora crassispinosa; Oxypora lacera; Pavona bipartita; Pavona venosa; Pectinia alcicornis; Pectinia paeonea; Platygyra acuta; Platygyra pini; Platygyra sp “green”; Platygyra verweyi; Podabacia cf lanakensis; Porites annae; Porites cylindrica; Porites evermanni; Porites monticulosa; Psammocora digitata; Psammocora explanulata; Psammocora haimeana; Psammocora superficialis; Sandalolitha dentata; Seriatopora caliendrum; Stylocoeniella armata; Stylocoeniella guentheri; Stylaster sp.; Tubipora musica; Turbinaria stellulata; Stylophora contorta; Stylophora danae; Stylophora kuehlmanni; Stylophora madagascarensis; Stylophora mamillata; Stylophora pistillata; Stylophora subseriata; Stylophora wellsi, or any coral known in the art, or a combination thereof.

Coral exoskeleton is typically porous. In some embodiments, the calcium carbonate-containing material comprises coral exoskeleton having an average pore size (e.g., average diameter) in the range of from 1 micron to 1 millimeter. In one embodiment, the average pore size of a coral ranges from 1 to 200 microns. In one embodiment, the average pore size of a coral ranges from 30 to 180 microns. In one embodiment, the average pore size of a coral ranges from 50 to 500 microns. In one embodiment, the average pore size of a coral ranges from 150 to 220 microns. In one embodiment, the average pore size of a coral ranges from 250 to 1000 microns.

For most therapeutic applications, it is desirable that the calcium carbonate-containing material, when derived from natural sources, such as coral, be devoid of any cellular debris or other organisms associated therewith in its natural state. Thus, in some of any of the embodiments described herein, the coral exoskeleton is an acellular coral exoskeleton.

Calcium carbonate-containing material such as, for example, aragonite, may be a commercially-available material or can be prepared from coral or coral fragments, or from coral sand. Briefly, the coral can be prepared as follows: in one embodiment, coral or coral sand is purified from organic residues, washed, bleached, frozen, dried, sterilized and/or a combination thereof prior to use in the compositions and/or methods of the present embodiments.

In some of any of the embodiments described herein, preparation of the aragonite or coral exoskeleton includes contacting solid coral exoskeleton of a desired size and shape with a solution comprising an oxidizing agent and washing and drying the solid material.

In some of any of the embodiments described herein, the oxidizing agent may be any suitable oxidizing agent, which facilitates the removal of organic debris from the coral exoskeleton. In some embodiments, the oxidizing agent is sodium hypochlorite.

According to this aspect, and in some embodiments, the process comprises conducting the contacting under mildly acidic conditions, so as to remove organic residues and provide acellular coral exoskeleton.

The calcium carbonate-containing material according to some embodiments of the present invention can be provided in a variety of forms, shapes and structures, compatible with a desired application. Some suitable forms and shapes include, but are not limited to, layers, blocks, spherical and hollow spherical forms, concentric spheres, rods, sheets, symmetrical and asymmetrical forms, amorphous and other irregular shapes and particles. The calcium carbonate-containing material can be shaped, for example, to fit a cavity or surface of tissue, or to fit an article containing the composition as described in further hereinafter.

In some of any of the embodiments described herein, the calcium carbonate-containing material (according to any of the respective embodiments and any combination thereof) is provided as particulate calcium carbonate-containing material.

In some embodiments, the particulate material comprises particles having an average particle diameter in the range of from 0.1 micron to 10 millimeter, or from 0.1 micron to 1 millimeter, or from 0.1 micron to 500 microns, or from 0.5 microns to 500 microns, or from 1 micron to 500 microns, or from 5.0 microns to 500 microns, including any subranges and intermediate values therebetween.

In some of any of the embodiments described herein, a calcium carbonate-containing material is produced from coral or coral sand according to a process comprising washing ground solid calcium carbonate (e.g. aragonite), such as coral or naturally occurring coral sand with water to desalinate it, then disinfecting and drying the desalinated coral sand at temperatures of about 80 degrees to about 150 degrees C., preferably 90 degrees to 120 degrees C., cutting larger pieces of coral into small pieces, and grinding the disinfected and dried coral or coral sand into particles of a desirable average size. In some embodiments, grinding is into particles of a size ranging from 5 to 500 microns.

In some embodiments, coral is ground into particles having a particle diameter of in the range of 1-5, 1-20, 1-50, 1-100, 5-10, 10-15, 15-20, 10-50, 10-100, 20-100, 50-100, 80-150, 100-200, 100-350, 150-500, 1-50 and/or 50-200 microns across, including any intermediate values and subranges therebetween. In some of any of these embodiments, coral is ground to particles having an average particle volume in the range of 1-100, 50-500, 250-1000, 500-2500, 1000-5000 and 2500-10,000 cubic micron or 0.01-0.1, 0.05-0.5, 0.5-0.75, 0.75-1.0, 1.0-2.0 and 1.0-5.0 cubic millimeters in volume, including any intermediate values and subranges therebetween.

In some of any of the embodiments described herein, the calcium carbonate-containing particulate material, including ACC and/or CS and/or any other material as described herein, comprises particles having a relatively small average particle diameter, for example, in a range of from 0.1 micron to 100 microns, or from 0.1 microns to 50 microns, including any intermediate values and subranges therebetween.

In some of any of the embodiments described herein, the calcium carbonate-containing particulate material, including ACC and/or CS and/or any other material as described herein, comprises particles having a relatively large average particle diameter, for example, an average diameter higher than 50 microns, for example, in the range of from 50 microns to 10 millimeter, or from 50 microns to 1 millimeter, or from 100 microns to 1 millimeter.

In some of any of the embodiments described herein, the calcium carbonate-containing particulate material, including ACC and/or CS and/or any other material as described herein, comprises a mixture of particles having a relatively large average particle diameter, as described herein, and particles having a relatively small average particle diameter, as described herein.

Exemplary calcium carbonate-containing materials are described in the Examples section that follows.

### The Citrate:

As used herein, the term “citrate salt” describes a compound composed of a citrate ion and one or more cations. The citrate ion can be represented by the formula C₆H₅O₇³⁻ or C₃H₅O(COO)₃³⁻. The cation can be monovalent, divalent or trivalent cation, and the stoichiometry of the citrate ion is in accordance with the selected cation.

The cation can be Na⁺, K⁺, Li⁺, Mg⁺², Zn⁺², Fe⁺², Fe⁺³, and any other suitable cation. If the cation is a monovalent cation, such as, for example, sodium cation, the citrate salt comprises 3 cations, and is, for example, tri-sodium citrate.

In some of any of the embodiments described herein, other salts of multicarboxylic acids can be used as alternative, or in addition, to a citrate salt as described herein.

By “multicarboxylic acid” it is meant an organic compound featuring two, three or more carboxylic acid groups. For a non-limiting example, a multicarboxylic acid can be represented by R(COOH)n, with R being an alkyl, alkenyl, cycloalkyl, and/or aryl, and n being an integer of at least 2 (e.g., 2, 3, 4, 5, etc.). The alkyl, alkenyl, cycloalkyl, or aryl, can be further substituted by one or more other substituents, as described herein.

In some of any of the embodiments described herein, other calcium-chelating agents can be used as alternative, or in addition, to a citrate salt as described herein.

In some of any of the embodiments described herein, other anti-coagulants can be used as alternative, or in addition, to a citrate salt as described herein. In some embodiments, such anti-coagulants are those acting by effecting the formation of cross-linked fibrin. In some embodiments, such anti-coagulants are not acting by effecting platelet aggregation. In some embodiments, the anti-coagulant is other than heparin or similarly-acting anti-coagulants that effect platelet aggregation.

### The Associating Moiety:

The associating moiety is aimed at associating the calcium carbonate-containing material and the citrate so as to form a composite material.

As discussed hereinabove, and without being bound to any particular theory, it is assumed that the associating moiety is such that can form physical and/or chemical interactions with one or both of the calcium carbonate-containing material and the citrate.

In some embodiments, the associating moiety is such that can form electrostatic interactions with one or more of the calcium carbonate-containing material and/or the citrate.

In some embodiments, the associating moiety features functional groups that are positively charged or negatively charged at physiological pH. Positively charged groups can form electrostatic interactions with the citrate and/or the carbonate, the latter leading to release of calcium ions. Negatively charged groups can for electrostatic interactions with calcium ions.

In some of any of the embodiments described herein, the associating moiety comprises one or more positively charged groups, and may further comprise one or more negatively charged groups.

According to some of any of the embodiments described herein, the association moiety is a biocompatible moiety.

In exemplary embodiments, the associating moiety is a positively-charged moiety at physiological pH.

The associating moiety can be a polymeric moiety or a non-polymeric moiety.

In some embodiments, the associating moiety is a polymeric moiety.

The polymeric moiety can be a large polymeric moiety, having a molecular weight higher than 100 kDa, or higher than 200 kDa, or 300 kDa or higher than 300 kDa, for example, in a range of from about 100 to about 1000, or from about 200 to about 1000, or from about 300 to about 1000, or from about 300 to about 800, or from about 300 to about 600, kDa, including any intermediate values and subranges therebetween.

The polymeric moiety can be a large polymeric moiety, having a molecular weight of 100 kDa or lower, for example, in a range of from about 10 to about 100, or from about 20 to about 100, or from about 30 to about 100, or from about 30 to about 80, or from about 30 to about 60, kDa, including any intermediate values and subranges therebetween.

Whenever a molecular weight or MW is described herein in the context of polymeric moieties (e.g., polypeptides), it is meant an average molecular weight, typically determined by conventional methods known in the art and/or in accordance with an information provided by the vendor thereof.

In some of any of the embodiments described herein, the associating moiety is a polymeric moiety, preferably a biocompatible polymeric moiety. In exemplary embodiments, the associating moiety is a polypeptide, featuring high or low molecular weight as described herein.

The term “polypeptide” as used herein encompasses native peptide macromolecules, including degradation products, synthetically prepared peptides and recombinant peptides (e.g., recombinantly expressed in a microorganism), as well as peptidomimetic macromolecules (typically, synthetically synthesized peptides), as well as peptoid and semipeptoid macromolecules which are peptide analogs, which may have, for example, modifications rendering the polypeptides more stable. Such modifications include, but are not limited to N-terminus modification, C-terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided herein below.

Peptide bonds (—CO—NH—) within the polypeptide may be substituted, for example, by N-methylated amide bonds (—N(CH₃)—CO—), ester bonds (—C(═O)—O—), ketomethylene bonds (—CO—CH₂—), sulfinylmethylene bonds (—S(═O)—CH₂—), α-aza bonds (—NH—N(R)—CO—), wherein R is any alkyl (e.g. methyl), amine bonds (—CH₂—NH—), sulfide bonds (—CH₂—S—), ethylene bonds (—CH₂—CH₂—), hydroxyethylene bonds (—CH(OH)—CH₂—), thioamide bonds (—CS—NH—), olefinic double bonds (—CH—CH—), fluorinated olefinic double bonds (—CF—CH—), retro-amide bonds (—NH—CO—), peptide derivatives (—N(R)—CH₂—CO—), wherein R is the “normal” side chain, naturally present on the carbon atom.

These modifications can occur at any of the bonds along the polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some of any of the embodiments described herein may also include one or more modified amino acids or one or more non-amino acid monomers e.g. fatty acids, complex carbohydrates, etc.

The term “amino acid” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The polypeptides of some embodiments of the invention are preferably utilized in a linear form, although it will be appreciated that in cases where cyclization does not severely interfere with polypeptide characteristics, cyclic forms of the polypeptide can also be utilized.

The polypeptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing polypeptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final polypeptide compound. By simple modification of this general procedure, it is possible to add more than one amino acid at a time to a growing chain, for example, by coupling (under conditions which do not racemize chiral centers) a protected tripeptide with a properly protected dipeptide to form, after de-protection, a pentapeptide and so forth. Further description of polypeptide synthesis is disclosed in U.S. Pat. No. 6,472,505. Large scale polypeptide synthesis is described, for example, by Andersson [Biopolymers 2000; 55 (3): 227-50].

Polypeptides of the present invention can be purified using a variety of standard protein purification techniques, such as, but not limited to, heat treatments, salting out for example with ammonium sulfate, polyethyleneimines (PEI) precipitation, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

According to some of any of the embodiments described herein, the associating moiety is a polypeptide, as described herein, and the polypeptide comprises one or more amino acid residue(s) that is/are positively charged at physiological pH.

Such amino acid residues typically comprise a primary or secondary amine group at the side chain thereof, and include, for example, the naturally occurring L-lysine, L-arginine, and L-histidine, and non-naturally occurring amino acid analogs thereof, such as, for example, D-lysine, D-arginine, D-histidine, ε-Lysine and ornithine.

In some of these embodiments, at least 5%, or at least 10%, or at least 20%, or at least 30%, or at least 40%, or at least 50%, or more, or substantially all, of the amino acid residues in the polypeptide are positively charged at physiological pH. The positively charged amino acid residues can be dispersed randomly within the polypeptide, and can include one or more types of positively charged amino acid residues, such as described herein.

In some of these embodiments, the polypeptide is consisted of amino acid residues that are positively charged amino at physiological pH, and can be, for example, polylysine, polyhistidine, polyarginine, polyornithine, etc.

In exemplary embodiments, the polypeptide comprises a plurality (e.g., at least 20%, or at least 50%, or at least 80%, or 100%) of lysine residues, which can be L-lysine residues and/or D-lysine residues. Alternatively, or in addition, the lysine residues are ε-lysine residues.

In exemplary embodiments, the polypeptide comprises a plurality (e.g., at least 20%, or at least 50%, or at least 80%, or 100%) of arginine residues, which can be L-arginine residues and/or D-arginine residues.

In exemplary embodiments, the polypeptide comprises a plurality (e.g., at least 20%, or at least 50%, or at least 80%, or 100%) of histidine residues, which can be L-histidine residues and/or D-histidine residues.

In exemplary embodiments, the polypeptide comprises a plurality (e.g., at least 20%, or at least 50%, or at least 80%, or 100%) of ornithine residues, which can be L-ornithine residues and/or D-ornithine residues.

In exemplary embodiments, the polypeptide is poly-D-lysine (PDL).

In exemplary embodiments, the polypeptide is poly-L-lysine (PDL).

Other polypeptides are also contemplated, for example, collagen (e.g., Types I, II and III), preferably human collagen, which can be synthetically prepared, recombinant, or extracted from a natural source. In exemplary embodiments, the collagen has average molecular weight (MW) that ranges from about 100 to about 200 kDa.

Any of the polypeptides described herein can have a low or high molecular weight as described herein.

According to some of any of the embodiments described herein, the associating moiety is a non-polymeric moiety.

The non-polymeric moiety can be, for example, positively charged at physiological pH.

The non-polymeric moiety can be, for example, an amino acid that is positively charged at physiological pH, as described herein in any of the respective embodiments.

In exemplary embodiments, the amino acid is L-lysine and/or D-lysine.

In exemplary embodiments, the amino acid is arginine, histidine, ornithine or ε-lysine.

In some embodiments, the associating moiety is capable of interfering in (e.g., inhibiting) a fibrinolysis process in a subject. Exemplary such associating moieties are positively charged polymeric and/or non-polymeric moieties as described herein (e.g., polypeptides and/or amino acids), and/or moieties that structurally resemble coagulants such as tranexamic acid or aminocaproic acid.

According to exemplary embodiments, the associating moiety is poly-D-lysine, poly-L-lysine, poly-D-L-lysine (with any ratio of the D-lysine and L-lysine), each having a high or low molecular weight as described herein, or any mixture thereof.

According to exemplary embodiments, the associating moiety is D-lysine, L-lysine, or a mixture thereof.

### The Composite Material:

The composite material of the present embodiments comprises the citrate, calcium-carbonate-containing material and the associating moiety, associated to one another, and encompasses any form of association, as described herein, between these components, and at any order.

In an exemplary configuration, at least a portion of the association moiety is deposited onto at least a portion of the surface of the calcium carbonate-containing material. In some of these embodiments, the calcium carbonate-containing material is a particulate material and the association moiety is deposited on a portion of the surface or practically coats the surface of at least a portion or all of the calcium carbonate-containing material particles.

In some of these embodiments, at least a portion of the calcium carbonate-containing material is associated with at least a portion of the association moiety via electrostatic interactions formed between the carbonate of the calcium carbonate-containing material and a positively charged group of the association moiety.

Further in this exemplary configuration, at least a portion of the citrate is associated with the portion of association moiety which is deposited onto a surface of the particulate material. Alternatively, or in addition, the citrate is associated with the calcium carbonate-containing material as described herein.

In some of these embodiments, at least a portion of the citrate is associated with at least a portion of the association moiety via electrostatic interactions and/or hydrogen bond interactions.

In some of any of the embodiments described herein, a weight ratio of the citrate and the calcium carbonate-containing material ranges from 10:1 to 1:10, or from 5:1 to 1:5, including any intermediate values and subranges therebetween, and can be, for example, 10:1, 5:1, 2:1, 1:1, 1:2, 1:5 or 1:10.

In some of any of the embodiments described herein, a weight ratio of the associating moiety and the calcium carbonate-containing material ranges from 5000:1 to 250:1, or from 2500:1 to 250:1, including any intermediate values and subranges therebetween, and can be, for example, 5000:1, 2500:1, 1000:1, 500:1 or 250:1.

The composite material may further comprise one or more additional components which may complement its function, such as antibiotics.

Preferably, the additional components are selected such as they do not interfere with the association and performance of the citrate, the calcium carbonate-containing material and the associating moiety.

In exemplary embodiments, the composite material may further comprise collagen, which is in association with the calcium carbonate, the associating moiety and/or the citrate.

According to an aspect of some embodiments of the present invention there is provided a composite material that comprises a calcium carbonate-containing material as described herein in any of the respective embodiments and any combination thereof, and an associating moiety as described herein in any of the respective embodiments and any combination thereof.

### Compositions

The composite material described herein can be used per se, or can be formulated together with a pharmaceutically acceptable carrier, to form a composition, e.g., a pharmaceutical composition.

As used herein, the term “pharmaceutically acceptable carrier” describes a carrier or a diluent that is used to facilitate the administration of the composite material (also referred to in this context as an active ingredient or active agent) or of the composition containing same and which does not cause significant irritation to an organism and does not abrogate the biological activity and properties of the administered active materials. Examples, without limitations, of carriers include water, buffered aqueous solutions, propylene glycol, emulsions and mixtures of organic solvents with water, as well as solid (e.g. powdered or polymeric) and gaseous carriers.

Techniques for formulation and administration of drugs may be found in “Remington's Pharmaceutical Sciences” Mack Publishing Co., Easton, PA, latest edition, which is incorporated herein by reference.

Compositions for use in accordance with the present embodiments thus may be formulated in conventional manner using one or more pharmaceutically acceptable carriers, excipients and/or auxiliaries, which facilitate processing of the compounds into preparations which can be used pharmaceutically. The dosage may vary depending upon the dosage form employed and the route of administration utilized.

The exact formulation, route of administration and dosage can be chosen by the individual physician in view of the patient's condition (see e.g., Fingl et al., 1975, in “The Pharmacological Basis of Therapeutics”, Ch. 1 p. 1).

The pharmaceutically acceptable carrier can be either an organic carrier or an aqueous carrier. In some embodiments, the carrier is an aqueous carrier. An aqueous carrier preferably comprises injectable-grade water, i.e., USP grade “water for injection”. However, other forms of purified water may be suitable, such as, for example, distilled and deionized water.

Aqueous formulations are preferred since these formulations are gentle to bodily tissues and are suitable for use on wounds. However, non-aqueous formulations are also contemplated. For example, in cases where the composition is in a form of a paste or an emulsion, non-aqueous carriers or mixed carriers of aqueous and organic carriers can be used.

The composition may be formulated for administration in either one or more of routes, depending on the area to be treated.

According to some embodiments, the composition is formulated for topical application, as a topical dosage form.

As used herein, the phrase “topical dosage form” describes a dosage form suitable for topical administration to the treated area (e.g., wound). By “topical administration” it is meant application onto the treated skin area, or “local administration”.

The compositions described herein can be, for example, in a form of a powder, granules, a cream, an ointment, a paste, a gel, a lotion, a milk, a suspension, an aerosol, a spray, a foam, a gauze, a wipe, a sponge, a wound dressing, a pledget, a patch, a pad, an adhesive bandage, and a non-adhesive bandage.

According to some embodiment, the composite is formulated in the form of a powder, a gel, a spray, a foam, a mousse, an ointment, a paste, a lotion, a gauze, a wound dressing, a suspension, an adhesive bandage, a non-adhesive bandage, a wipe, a gauze, a pad, and a sponge

In some embodiments, the composition is formulated as a liquid reservoir, to be applied as drops, spray, aerosol, liquid, foam and the like. Suitable carriers and other ingredients are used in these cases. For example, for application as an aerosol or foam, a propellant is used. For application as foam, foam-forming agents can also be used.

In some embodiments, the composition is formulated as a cream. Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information. An exemplary cream formulation can be obtained by mixing the composite material described herein with a carrier comprising cellulose derivatives such as cellulose acetate, hydroxyethyl cellulose and/or a polyethylene glycol.

In some embodiments, the composition is formulated as an ointment. Ointments are semisolid preparations, typically based on petrolatum or petroleum derivatives. The specific ointment base to be used is one that provides for optimum delivery for the active agent chosen for a given formulation. As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

In some embodiments, the composition is formulated as a lotion. Lotions are preparations that are to be applied to the skin surface without friction. Lotions are typically liquid or semiliquid preparations in which solid particles, namely, the calcium carbonate-containing material particles, are present in a water or alcohol base. Lotions are typically preferred for covering/protecting large body areas, due to the case of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

In some embodiments, the composition is formulated as a paste. Pastes are semisolid dosage forms in which the bioactive agent is suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

In some embodiments, the composition is formulated as a gel. Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

In some embodiments, the composition is formulated as a foam. Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or hydroalcoholic, but are typically formulated with high alcohol content which, upon application to the treated area, quickly evaporates, driving the composite material to the site of treatment.

In some embodiments, the composition is formulated as a powder or granules. Such compositions can optionally be prepared by preparing the composite material (e.g., as described herein) and forming granules or beads containing these ingredients, for example, by adding suitable agents (e.g., water soluble film-forming agents).

In some embodiments, a topical dosage form includes a solid or semi-solid substrate, e.g., a gauze, a wipe, a bandage, a pad, a pledget, a sponge, a mesh, a fabric, and the likes, and the composite material is incorporated in and/or on the substrate.

The substrate in such topical dosage forms can be of any form and materials used to make up gauzes, wipes, bandages, pads, pledgets, sponges, meshes, fabrics (woven and non-woven, cotton fabrics, and the like), and any other substrates commonly used in medical applications.

Such topical dosage forms may optionally further comprise an adhesive, for facilitating the topical application of the composition onto the treated area for a prolonged time period.

Exemplary adhesives include, but are not limited to, medically acceptable bioadhesives, polymer glues, etc., and can be applied to the substrate by, for example, dip coating with an adhesive base. Such dip coating can be effected during manufacture of the substrate, or at any time prior to its application. In some embodiments, the composite material can be embedded within and/or on the material of the substrate, for example, embedded into or onto a polymer or fabrics by application of heat, or fused to the substrate. In other embodiments, the composite material can be incorporated into the base material of the substrate, for example, mixed within the components of a polymer before polymerization, or mixed with components forming fibers used to make up a gauze or a mesh or pad, etc.

The composition described herein can further comprise additional ingredients, which are aimed at improving or facilitating its preparation, application and/or performance. Such additional ingredients include, for example, anti-irritants, anti-foaming agents, humectants, deodorants, antiperspirants, preservatives, emulsifiers, occlusive agents, emollients, thickeners, penetration enhancers, colorants, propellants and/or surfactants, depending on the final form of the composition.

Representative examples of humectants that are usable in this context of the present embodiments include, without limitation, guanidine, glycolic acid and glycolate salts (e.g. ammonium slat and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

Representative examples of deodorant agents that are usable in the context of the present embodiments include, without limitation, 2,4,4′-trichloro-2′-hydroxy diphenyl ether, and diaminoalkyl amides such as L-lysine hexadecyl amide.

Suitable preservatives that can be used in the context of the present embodiments include, without limitation, one or more alkanols, parabens such as methylparaben and propylparaben, propylene glycols, sorbates, urea derivatives such as diazolindinyl urea, or any combinations thereof.

Suitable emulsifiers that can be used in the context of the present embodiments include, for example, one or more sorbitans, alkoxylated fatty alcohols, alkylpolyglycosides, soaps, alkyl sulfates, or any combinations thereof.

Suitable occlusive agents that can be used in the context of the present embodiments include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients, that can be used in the context of the present embodiments include, for example, dodecane, squalane, cholesterol, isohexadecane, isononyl isononanoate, PPG ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Suitable thickeners that can be used in the context of the present embodiments include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, and mixtures thereof.

Suitable penetration enhancers usable in context of the present embodiments include, but are not limited to, polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, menthol, TWEENS such as TWEEN 20, and the like. The permeation enhancer may also be a vegetable oil. Such oils include, for example, safflower oil, cottonseed oil and corn oil.

Suitable anti-irritants that can be used in the context of the present embodiments include, for example, steroidal and non-steroidal anti-inflammatory agents or other materials such as menthol, aloe vera, chamomile, alpha-bisabolol, cola nitida extract, green tea extract, tea tree oil, licorice extract, allantoin, caffeine or other xanthines, glycyrrhizic acid and its derivatives.

Any of the additional ingredients or agents described herein is preferably selected as being compatible with the components of the composite material as described herein, such that there is no interference with the availability of these materials in the composition.

Any of the additional ingredients described herein is further preferably selected as being biocompatible.

In some embodiments, the composition further comprises an additional therapeutically active agent, for example, an additional hemostatic agent or composition or article, or, for example, an agent capable of disinfecting the treated area (e.g., antiseptic agents or compositions).

Compositions of the present embodiments may, if desired, be presented in a pack or dispenser device, such as an FDA (the U.S. Food and Drug Administration) approved kit, which may contain one or more unit dosage forms containing the composite material. The pack may comprise, for example, glass or plastic foil. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions of the invention formulated in a compatible pharmaceutical carrier may also be prepared, placed in an appropriate container, and labeled for a medical indication, as detailed herein.

The compositions described herein may be packed or presented in any convenient way. For example, they may be packed in a tube, a bottle, a dispenser, a squeezable container, or a pressurized container, using techniques well known to those skilled in the art and as set forth in reference works such as Remington's Pharmaceutical Science 15^{th} Ed. It is preferred that the packaging is done in such a way so as to minimize contact of the unused compositions with the environment, in order to minimize contamination of the compositions before and after the container is opened.

The compositions described herein are preferably supplied in the concentration intended for use but may also be prepared as concentrates that are diluted prior to use. For example, concentrates requiring dilution ratios of 2:1 to 10:1 parts carrier to a concentrate are contemplated.

In some embodiments, the composition described herein is packaged in a packaging material and identified in print, in or on the packaging material, for use in treating chronic wounds, nociceptive pain or non-bleeding wounds, as described herein.

### Articles-of-Manufacturing:

According to an aspect of some embodiments of the present invention, an article-of-manufacturing is provided, which comprises the composite material or the composition as described herein in any of the respective embodiments, and any combination thereof, and means for topically applying the composite material or a composition comprising same onto the treated area. In some embodiments, the article-of-manufacturing is configured to apply the composition to wounds.

In some embodiments, the article-of-manufacturing comprises the composition as described herein, in a form of a suspension, packaged in a container, and means for applying the composition as drops, spray, aerosol, foam, using techniques well known to those skilled in the art and as described herein.

In some embodiments, the article-of-manufacturing comprises the composition as described herein, in a form of a cream, lotion, paste, ointment, and the likes, packaged in a suitable container, and optionally comprising means for dispensing the composition from the container.

In some embodiments, the article-of-manufacturing comprises the composite material or a composition comprising same as described herein, in a form that comprises a powder or granules, packaged in a suitable container, and optionally comprising means for dispensing the composition from the container.

In some embodiments, the article-of-manufacturing comprises the composite material or a composition comprising same as described herein, incorporated in and/or on a substrate, as described herein. The article-of-manufacturing can be packaged in a sterile packaging. In exemplary embodiments, the substrate is a gauze or any other solid substrate usable in medical applications, and the article-of-manufacturing is bandage comprising the composite material.

The article-of-manufacturing can be labeled as described herein, for example, by being identified in print, in or on the packaging material, for use in treating chronic wounds, nociceptive pain or non-bleeding wounds.

### Kits

According to an additional aspect of embodiments of the invention there is provided a kit, which comprises the composite material or a composition comprising same as described herein, being packaged in a packaging material.

The kit can be labeled, for example, by being identified in print, in or on the packaging material, for use in treating chronic wounds, nociceptive pain or non-bleeding wounds.

The components of the composition can be packaged within the kit either together, as a single, ready for use, composition, or at least one of the components (e.g., a carrier or a solid substrate) can be packaged individually. When one or more components are packaged individually, the kit may further be supplied with instructions indicating the route of to prepare and/or apply the components so as to contact an area to be treated with the composite material or the composition. Such instructions can be, for example, mixing the components (e.g., the composite material and the carrier) prior to application or simultaneously or subsequently applying the components (e.g., the composite material and the carrier) onto an area to be treated.

In an exemplary embodiment, the kit comprises the composite material in a first container (e.g., a sterile packaging), and a liquid carrier packaged individually (e.g., in another container), and instructions to add the carrier to the first container, prior to application of the composition to the area to be treated, or vice versa (instructions to add the content of the first container to the carrier in the second container). The first (or second) container can be configured to apply the composition as drops, spray, aerosol, foam, etc. The kit may alternatively further comprise a device for dispensing the composition.

In another exemplary embodiment, the kit comprises the composite material in a first container (e.g., a sterile packaging), and a solid carrier (e.g., a gauze) packaged individually (e.g., in another container), and instructions to contact the composite material with the solid carrier to provide a composition, prior to application of the composition to the area to be treated or instructions to contact a wounded area to be treated with the composite material and apply the gauze or other solid substrate onto the applied composite material. The kit may alternatively further comprise a device for dispensing the composite material.

In another exemplary embodiment, the kit comprises the calcium carbonate-containing material, the association moiety and the citrate, each packaged individually, and each optionally together with a carrier, or a carrier is optionally packaged individually with the kit. The carrier is such that is suitable for the selected dosage form, as described herein. The kit further comprises instructions to prepare the composite material, as described herein, and optionally mix the composite material with the carrier or otherwise use the composite material in combination with the carrier as described herein.

The containers, substrates, and compositions included in the kit can be in accordance with any of the embodiments described herein, and any combination thereof.

### Treatment Regimen

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. The dosage, schedule and duration of treatment can vary, depending on the severity of pathology and the selected type of treatment, and those of skills in the art are capable of adjusting the type of treatment with the dosage, schedule and duration of treatment.

According to some embodiments, the regimen includes multiple administrations.

Thus, for instance, the composite or composition comprising same ban be administered once a day or a number of times a day (e.g., at least 2, 3, 4) for a plurality of days, e.g., at least 3, 7, 10, 14, 21, 30 days, one month, two months, 3 months, 4 months, 5 months, 6 months, 7 months, 8 months, 9 months, 10 months, 11 months, 12 months, 18 months, 24 months or more. Alternatively, the composite or composition comprising same ban be administered once a day or a number of times a day (e.g., at least 2, 3, 4) for a plurality of days but dependent on the severity. Hence there can be periods, e.g., 2, 3, 7, 10 days in which the subject does not treat with the composite or composition comprising same and treatment may be resumed as needed.

According to a specific embodiment, the multiple administrations are effected over a period of at least 7 days.

It will be appreciated that a single treatment or an acute treatment not exceeding 2, 3 days is also contemplated such as for the treatment of nociceptive pain.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements. Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### CPC Causes Healing of Chronic Wounds in Horses

### Materials and Methods

### Preparation of CPC

For preparation of 100 mg CPC: 100 g r ACC and 40 mg lysine were added to 250 ml double distilled water (DDW) and incubated over night (O.N.) at 4° C. The sample was then centrifuged for 3 minutes (1000 g, 4° C.). The supernatant was removed and the wash was repeated twice more. The last supernatant was removed and 200 ml of DDW with 6.4 mg sodium citrate were added to reach a final citrate concentration of 3.2%. After incubation for 5 min in room temperature the sample was centrifuged and washed as above, reloaded with 5 ml DDW and let dry in a biological hood for O.N. The powder was then aliquoted to 50 ml tubes and kept in 4° C. in a dissector.

Treatment with CPC

Treatment was provided by application of CPC directly on the wound. The amount used varied according to the wound size. Usually, extra amount was applied so to cover the entire injured area and its depth. Following application, the wound was covered with bandages and left for 1-4 days. Refreshment of the treatment was done by washing the old dressing with water, drying in air and reapplication. In some cases controls were done where Granuloflex was used instead of CPC.

### Wound Size Analysis

A ruler was positioned near the wound and images before and at different time points after the treatment were collected. The images were than sized to equalize their scale and analyzed using the NIH ImageJ software. The peripheral couture of each wound was drawn manually and the area it engulfed was collected as pixel number and converted to a calibrated meter scale. To calculate the rate of wound side decrease at each time point the following formula was used: Percent reduction (at time X)=[Area (time X)/Area (time 0−before treatment)]×100.

### Results

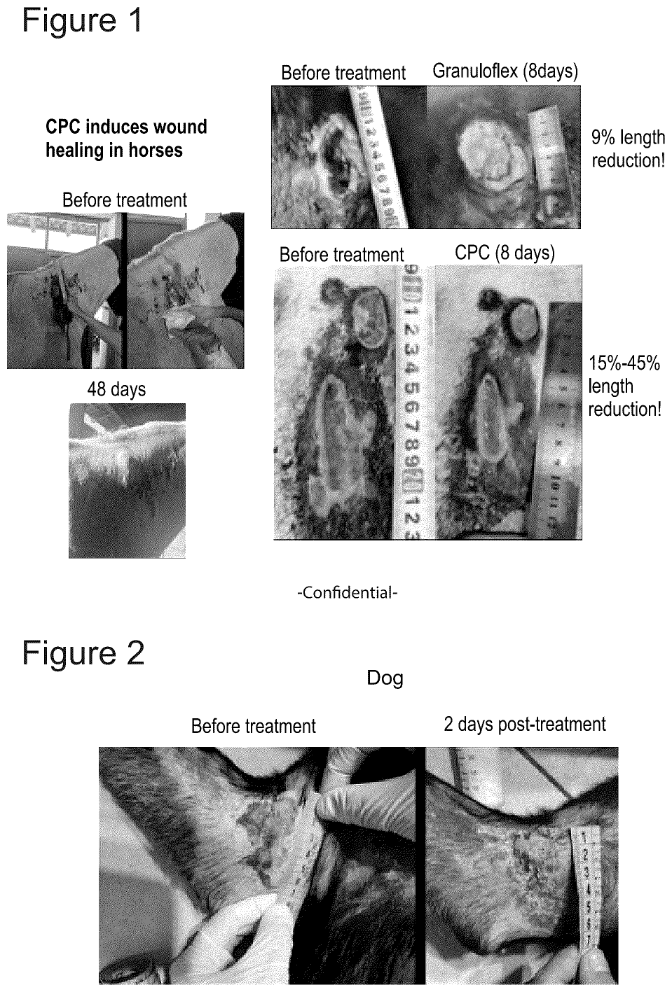

A first set of experiments was performed on chronic wounds in horses produced by injection of non-pathologic snake's poison for immunization and antibody production (Image 1). The wounds were few centimeters long and few millimeters deep. When treated with conventional moistures and ointments, such as Granuloflex and Dermadan, the wounds persisted for months and kept secreting. By contrast, and as shown in FIG. 1, when treated with CPC wounds became granulized within a day or two, stopped secreting, became dry, and their size shrunk during the first 3 weeks of treatment (FIG. 2) and disappeared completely in less than a month and a half (FIG. 1).

### Example 2

### CPC Causes Healing of Chronic Wounds in Dogs

### Materials and Methods

### Preparation of CPC

For preparation of 100 mg CPC: 100 g r ACC and 40 mg lysine were added to 250 ml double distilled water (DDW) and incubated over night (O.N.) at 4° C. The sample was then centrifuged for 3 minutes (1000 g, 4° C.). The supernatant was removed and the wash was repeated twice more. The last supernatant was removed and 200 ml of DDW with 6.4 mg sodium citrate were added to reach a final citrate concentration of 3.2%. After incubation for 5 min in room temperature the sample was centrifuged and washed as above, reloaded with 5 ml DDW and let dry in a biological hood for O.N. The powder was then aliquoted to 50 ml tubes and kept in 4° C. in a dissector.

Treatment with CPC

Treatment was provided by application of CPC directly on the wound. The amount used varied according to the wound size. Usually, extra amount was applied so to cover the entire injured area and its depth. Following application, the wound was covered with bandages and left for few days. Refreshment of the treatment was done by washing the old dressing with water, drying in air and reapplication.

### Wound Size Analysis

A ruler was positioned near the wound and images before and at different time points after the treatment were collected. The images were than sized to equalize their scale and analyzed using the NIH ImageJ software. The peripheral couture of each wound was drawn manually and the area it engulfed was collected as pixel number and converted to a calibrated meter scale. To calculate the rate of wound side decrease at each time point the following formula was used: Percent reduction (at time X)=[Area (time X)/Area (time 0−before treatment)]×100.

### Results

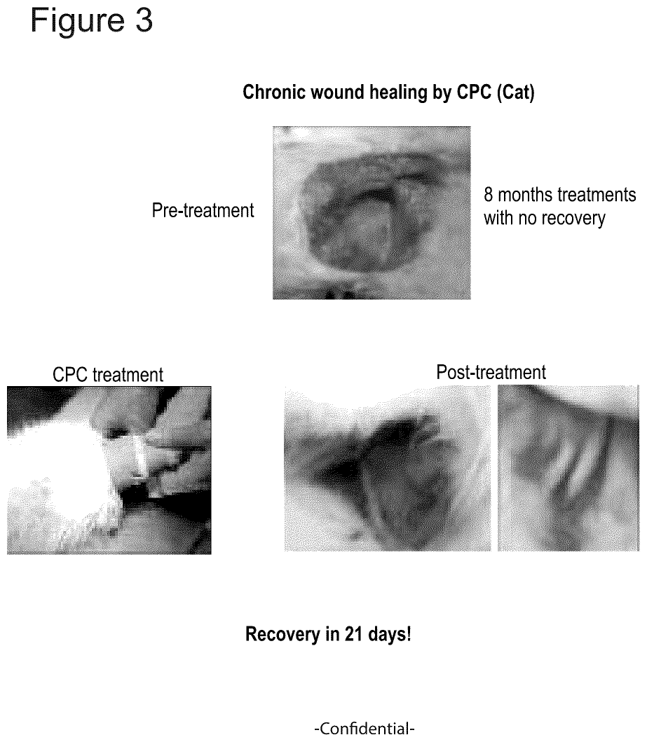

Another experiment was performed on a dog with a snake bite injury (FIG. 2). The dog was treated unsuccessfully with conventional treatments for two months and was behaviorally depressed, refusing to walk or eat. Following treatment with CPC the wound length shrunk by close to 50% in two days and continued in this trend in the following days (FIG. 3). The dog's behavior changed dramatically, as evidenced by walking and eating.

### Example 3

### CPC Causes Healing of Chronic WOUND in Domesticated Animals

### Materials and Methods

### Preparation of CPC

For preparation of 100 mg CPC: 100 g r ACC and 40 mg lysine were added to 250 ml double distilled water (DDW) and incubated over night (O.N.) at 4° C. The sample was then centrifuged for 3 minutes (1000 g, 4° C.). The supernatant was removed and the wash was repeated twice more. The last supernatant was removed and 200 ml of DDW with 6.4 mg sodium citrate were added to reach a final citrate concentration of 3.2%. After incubation for 5 min in room temperature the sample was centrifuged and washed as above, reloaded with 5 ml DDW and let dry in a biological hood for O.N. The powder was then aliquoted to 50 ml tubes and kept in 4° C. in a dissector.

Treatment with CPC

Treatment was provided by application of CPC directly on the wound. The amount used varied according to the wound size. Usually, extra amount was applied so to cover the entire injured area and its depth. Following application, the wound was covered with bandages and left for few days. Refreshment of the treatment was done by washing the old dressing with water, drying in air and reapplication.

### Wound Size Analysis

A ruler was positioned near the wound and images before and at different time points after the treatment were collected. The images were than sized to equalize their scale and analyzed using the NIH ImageJ software. The peripheral couture of each wound was drawn manually and the area it engulfed was collected as pixel number and converted to a calibrated meter scale. To calculate the rate of wound side decrease at each time point the following formula was used: Percent reduction (at time X)=[Area (time X)/Area (time 0-before treatment)]×100.

### Results

Experiments were performed in acute wound in a cat (FIG. 3). The wound was unsuccessfully treated by a vet for 8 months, whereas the CPC treatment caused recovery within a month.

### Example 4

### CPC Treats Non-Bleeding Skin Wounds in Mice

### Materials and Methods

Preparation of CPC—as in Example 3 above.

Mice treatment—Two months old mice were anesthetized using Ketamine; Xylasine 1:3. After removal of the feather holed (6 mm diameter, 1 mm deep) were made using punch and the skin removed. Treatment groups received 30 mg CLC at time 0 followed by tightening with gauze. Control animals received no treatment. Wounds were covered with transparent plaster. At 3 days following the injury the old CLC was replaced by a fresh one. The wounds were cleaned at days 7, 10 and 14, photographed and the wounds diameter was measured from the images using the ImageJ software.

### Results

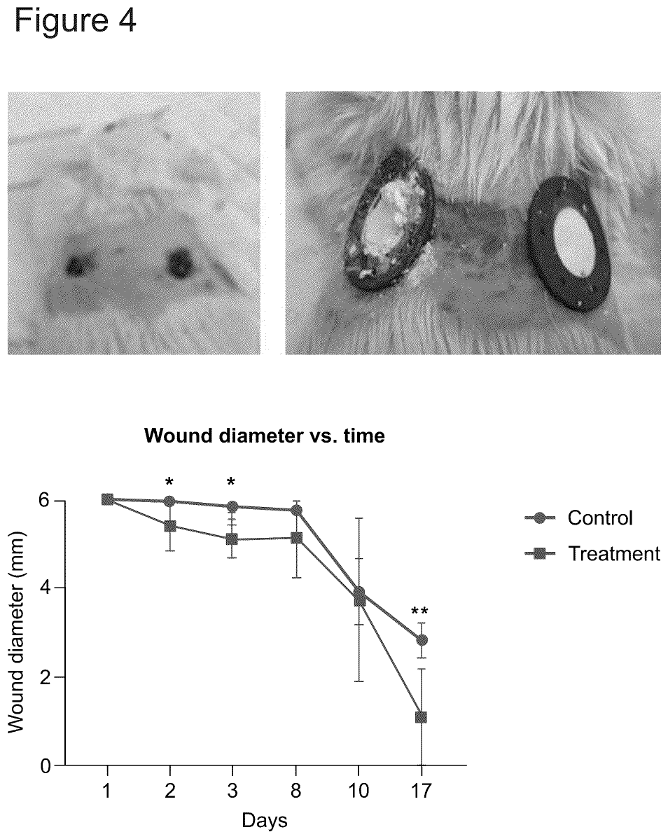

The capacity of CPC to heal a non-bleeding skin wound was detected also when using a skin injury model in mice. Mice were injured with a 6 mm punch followed by treatment with CPC or nothing as a control. A statistically significant reduction in wound diameter was detected in CPC treated mice vs the control (FIG. 4).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of treating a chronic wound, a non-bleeding wound or treating or preventing an inflammatory pain or a nociceptive pain in a subject in need thereof, the method comprising administering to the subject an effective amount of a composite material comprising a calcium carbonate-containing material, and an associating moiety being associated with said calcium carbonate-containing material.

2-7. (canceled)

8. The method of claim 1, wherein said wound is a skin wound or diabetic wound.

9-11. (canceled)

12. The method of claim 1, wherein said administering comprises topical administration.

13-15. (canceled)

16. The method of claim 1, wherein said composite further comprises a citrate in chemical and/or physical association with the calcium carbonate, and/or with the association moiety.

17. The method of claim 1, wherein said associating moiety is a positively-charged moiety at physiological pH.

18. The method of claim 1, wherein said association moiety is a polymeric moiety or wherein said association moiety has a molecular weight in a range of 10 to 100 kDa or wherein said association moiety has a molecular weight of at least 300 kDa or wherein said association moiety is a biocompatible moiety or wherein said association moiety is a polypeptide.

19-22. (canceled)

23. The method of claim 18, wherein said polypeptide comprises at least one amino acid residue that is positively charged at physiological pH or wherein said polypeptide essentially consists of amino acid residues that are positively charged at physiological pH.

24. (canceled)

25. The method of claim 18, wherein said polypeptide is or comprises a polylysine.

26. The method of claim 18, wherein said polylysine is selected poly-D-lysine, poly-L-lysine and poly-ε-lysine.

27. The method of claim 18, wherein said polypeptide is or comprises collagen.

28. The method of claim 1, wherein said association moiety is or comprises lysine.

29. The method of claim 28, wherein said lysine is selected from L-lysine or D-lysine.

30. The method of claim 1, wherein the calcium carbonate-containing material comprises crystalline calcium carbonate.

31. The method claim 1, wherein said calcium carbonate-containing material comprises a coral exoskeleton.

32. The method of claim 1, wherein the calcium carbonate-containing material comprises amorphous calcium carbonate (ACC) or calcite.

33. The method of claim 1, wherein said calcium carbonate-containing material is a particulate material.

34. The method of claim 33, said particulate material comprises particles having an average particle diameter in the range of from 0.1 micron to 10 millimeter, or from 0.1 micron to 1 millimeter, or from 0.1 micron to 500 microns, or from 0.5 microns to 500 microns, or from 1 micron to 500 microns, or from 5.0 microns to 500 microns.

35. The method of claim 33, wherein said particulate material comprises particles having an average particle diameter in the range of from 0.1 micron to 100 microns, or from 0.1 microns to 50 microns.

36. The method of claim 33, wherein said particulate material comprises particles having an average diameter in the range of from 100 microns to 10 millimeter, or from 100 microns to 1 millimeter.

37. The method of claim 16, wherein a weight ratio of said citrate and said calcium carbonate-containing material ranges from 10:1 to 1:10, or from 5:1 to 1:5.

38. The method of claim 1, wherein a weight ratio of said association moiety and said calcium carbonate-containing material ranges from 5000:1 to 250:1.

39. The method of claim 1, further comprising a swelling polymeric moiety.

40. The method of claim 39, wherein said swelling polymeric moiety is selected from alginate, chitosan, collagen and a poly(alkylene glycol).
